(19) United States
(12) Patent Application Publication
(10) Pub. No.: US 2019/0200843 A1
(43) Pub. Date: Jul. 4, 2019

(54) BENDING SECTION FOR DISPOSABLE ENDOSCOPE

(71) Applicant: Zhuhai Kaden Medical Imaging Technology Co., Ltd, Zhuhai (CN)

(72) Inventors: Kam Shu Lai, Zhuhai (CN); Nanbing Wang, Zhuhai (CN)

(21) Appl. No.: 15/954,652

(22) Filed: Apr. 17, 2018

(30) Foreign Application Priority Data
Jan. 2, 2018 (CN) 201810002693.6
Feb. 9, 2018 (CN) 201810133991.9

(51) Int. Cl. A61B 1/008; A61B 1/005

(52) U.S. Cl. CPC A61B 1/008; A61B 1/0057

(57) ABSTRACT

The present disclosure provides a bending section for a disposable endoscope. The bending section comprises a plurality of pull wire locating rings, which are made of a flexible material. Each of the pull wire locating rings has two opposing side walls defining a pull wire perforation therein respectively, the pull wire perforation running through the side wall. The pull wire locating rings are hinged end to end in series to form a bendable flexible tube structure. The pull wire perforations in the pull wire locating rings are communicated in sequence to define a pull wire channel located in the side walls of the pull wire locating rings. The pull wire locating ring has one end surface defining a notch. The present disclosure has the flexibility greatly enhanced and is more sensitive to pull.

### CROSS REFERENCES TO RELATED APPLICATION

This application claims priority of Chinese Patent Application Nos. 201810002693.6 filed on Jan. 2, 2018 and 201810133991.9 filed on Feb. 9, 2018. The entire contents of the above are hereby incorporated by reference.

### TECHNICAL FIELD

The present disclosure relates to the field of medical apparatus and instruments, and in particular to a bending section for a disposable endoscope.

### BACKGROUND

With the development of medicine, endoscopic surgeries are increasingly used in clinical practices in recent years. Endoscopic flexible tube is one of the important parts of an endoscope. The flexible tube is arranged in the front end of the endoscope to form a bending section, for facilitating a lens to change direction inside a human body. Current bending sections are made of metal materials and are fabricated by riveting or welding many pull wire locating rings, which are made of metal materials, one by one in sequence. Each pull wire locating ring has an outer wall defining a recess, and a pull wire is arranged to pass through these recesses in sequence. Since the pull wire locating ring is made of metal materials, the flexibility is not high enough and sharp-pointed rigid corrugations are easy to occur, which makes the pull wire pulled unsmoothly. Once one of the pull wire locating rings is damaged, the pull wire cannot be pulled, consequently, the entire bending section and even the entire endoscope must be replaced. On the other hand, since the metallic pull wire locating ring is riveted or welded to one another, the processing technique is complex and bad products are easy to appear. Moreover, during the usage process, once the riveting point or welding point falls off or is unsoldered, combined with the rigidness of the metal materials, the entire bending section is easy to fail in work, the pull wire cannot be pulled and the endoscope cannot deflect. Thus, inconvenience is caused to operators and unnecessary pain is caused to patients.

Thus, existing technologies have certain drawbacks.

### SUMMARY

In view of the above, in order to solve the problems in existing technologies, the present disclosure provides a bending section for a disposable endoscope, which is simple to process and is high in reliability.

The present disclosure solves the above problem by the following technical means.

A bending section for a disposable endoscope includes a plurality of pull wire locating rings, which are made of a flexible material.

Each of the pull wire locating rings has two opposing side walls defining a pull wire perforation therein respectively, the pull wire perforation running through the side wall.

The pull wire locating rings are hinged end to end in series to form a bendable flexible tube structure. The pull wire perforations in the pull wire locating rings are communicated in sequence to define a pull wire channel located in the side walls of the pull wire locating rings.

The pull wire locating ring has one end surface defining a notch. The notch is a gap reserved for two adjacent pull wire locating rings to be capable of rotating relative to each other.

Further, the two pull wire perforations on the pull wire locating ring have an axis line parallel to the axis line of the pull wire locating ring.

Further, the axis line of the pull wire locating ring is intersected with a connection line used for connecting the axis lines of the two pull wire perforations.

Further, each of the pull wire locating rings has one end surface provided with two clamping lugs convexly, and has the other end surface provided with two clamping lug seats convexly; the two clamping lugs are provided with a round rod respectively, and the two clamping lug seats define a through hole respectively; the round rods on the two clamping lugs of one pull wire locating ring are inserted into the through holes on the two clamping lug seats of one adjacent pull wire locating ring respectively; and the round rods and the through holes are in one-to-one correspondence, thereby realizing the hinging between the two adjacent pull wire locating rings.

Further, the end surface of the pull wire locating ring provided with clamping lugs defines a notch, and the end surface of the pull wire locating ring provided with clamping lug seats is a plane.

Further, the end surface of the pull wire locating ring provided with clamping lugs is a plane, and the end surface of the pull wire locating ring provided with clamping lug seats defines a notch.

Further, the notch is shaped as a concaved curved surface.

Further, the pull wire locating ring is made of a medical plastic.

Compared with existing technologies, the present disclosure has the following beneficial effects.

1. According to the present disclosure, a flexible material is employed to replace traditional metal materials. Meanwhile, a bending gap is reserved. Therefore, the bending section has the flexibility greatly enhanced and is more sensitive to pull.

2. Necessary processes for metal materials, such as welding and riveting, are abandoned. Shaping can be realized by mold injection directly. The assembling is simple and has high yield.

3. The present disclosure avoids the recess structure provided on the surface of the traditional metal bending section and arranged for a pull wire to pass through. The pull wire is arranged to directly pass through the side wall. Therefore, frictions caused by the pull wire frequently entering and exiting the outer wall of the bending section are greatly reduced. Since the pull wire locating ring is made of a flexible material, the pull wire locating ring is difficult to be damaged by frequent pulling during the usage process. The usage stability and reliability are significantly improved. On the other hand, even if one or more pull wire locating rings fall off or are damaged, the pulling easiness of the pull wire is not affected. The condition that the pull wire of the metal bending section is stuck in some pull wire locating ring is avoided.

### BRIEF DESCRIPTION OF THE DRAWINGS

To better describe the technical scheme in the embodiment of the present disclosure, accompanying drawings needed in the description of the embodiment are simply illustrated below. Obviously, accompanying drawings described hereinafter merely illustrate some embodiments of the present disclosure. For the ordinary skill in the field, other accompanying drawings may be obtained according to these accompanying drawings without creative work.

FIG. 1 is a structure diagram of a pull wire locating ring in the present disclosure.

FIG. 2 is a sectional view of a pull wire locating ring in the present disclosure.

FIG. 3 is a structure diagram of two adjacent pull wire locating rings that are in connection in the present disclosure.

FIG. 4 is a sectional view of two adjacent pull wire locating rings that are in connection in the present disclosure.

FIG. 5 is a structure diagram of the bending section when in a bending state.

### DESCRIPTION OF DESIGNATORS IN DRAWINGS

1 designates a pull wire locating ring, 2 designates a side wall, 3 designates a pull wire perforation, 4 designates a pull wire channel, 5 designates a notch, 6 designates a clamping lug, 7 designates a clamping lug seat, 8 designates a round rod, and 9 designates a through hole.

### DESCRIPTION OF THE EMBODIMENTS

The above purpose, technical scheme and advantages of the present disclosure will become more clearly understood from the detailed description of the technical scheme of the present disclosure below taken in conjunction with accompanying drawings and specific embodiments. It should be noted that the embodiments described hereinafter are simply part embodiments of the present disclosure, but all the embodiments. All other embodiments obtained by the ordinary skill in the art based on the embodiments in the present disclosure without creative work are intended to be included in the scope of protection of the present disclosure.

It is to be noted that directional or positional relations indicated by terms such as “top”, “bottom”, etc. are directional or positional relations shown based on the drawings, merely to conveniently describe the present disclosure and simplify the description, but to indicate or imply the designated device or element to be constructed and operated in a specific position or in a specific direction. Therefore, the used directional terms cannot be understood as a limit to the present disclosure.

Terms “first”, “second” and “third” are merely for the purpose of description, but cannot be understood as the indication or implication of relative importance or as the implicit indication of the number of the designated technical features. Therefore, features defined by “first”, “second” and “third” may explicitly or implicitly include one or more such features. In the description of the present disclosure, unless otherwise stated, “a group of” means two or more than two.

### Embodiment

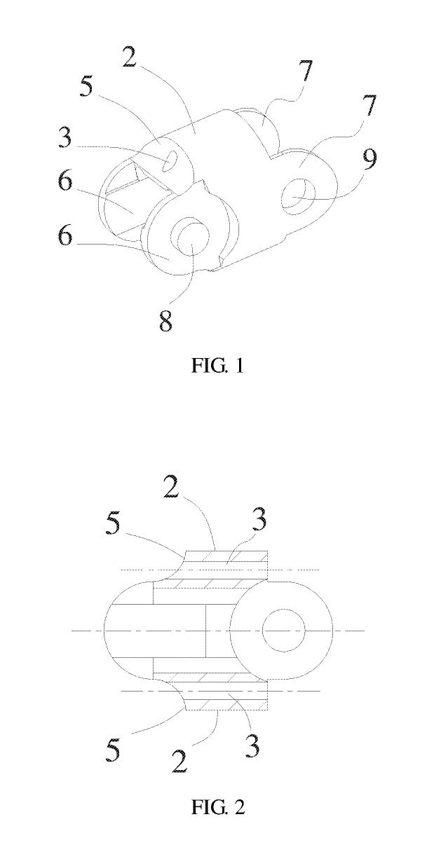

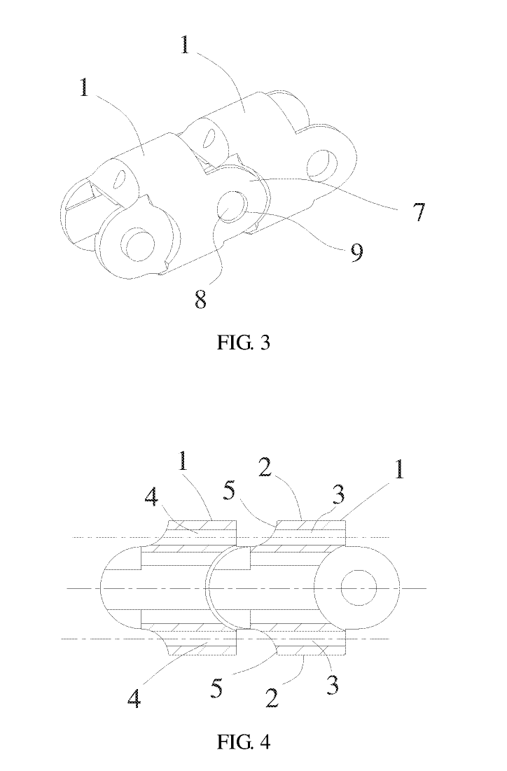

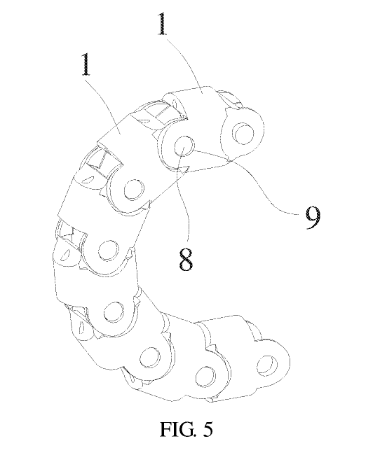

Referring to FIG. 1 to FIG. 5, a bending section for a disposable endoscope includes a plurality of pull wire locating rings 1, which are made of a flexible material.

Each of the pull wire locating rings 1 has two opposing side walls 2 defining a pull wire perforation 3 therein respectively, the pull wire perforation 3 running through the side wall 2.

The pull wire locating rings 1 are hinged end to end in series to form a bendable flexible tube structure. The pull wire perforations 3 in the plurality of pull wire locating rings 1 are communicated in sequence to define a pull wire channel 4 located in the side walls 2 of the pull wire locating rings 1. The two pull wire channels 4 accommodate a pull wire respectively.

The two pull wires have one respective end connected to the pull wire locating ring 1 located at the tail end of the flexible tube structure.

The pull wire locating ring 1 has one end surface defining a notch 5. The notch 5 is a gap reserved for two adjacent pull wire locating rings 1 to be capable of rotating relative to each other.

According to the present disclosure, the pull wire locating ring 1 employs a flexible material to replace traditional metal materials. Meanwhile, a notch 5 is defined and a bending gap is reserved. Therefore, the bending section has the flexibility greatly enhanced and is more sensitive to pull. Moreover, necessary processes for metal materials, such as welding and riveting, are abandoned. Shaping can be realized by mold injection directly. The assembling is simple and has high yield. Besides the above, the present disclosure avoids the recess structure provided on the surface of the traditional metal bending section and arranged for a pull wire to pass through. The pull wire is arranged to directly pass through the side wall 2 via the pull wire perforation 3. Therefore, frictions caused by the pull wire frequently entering and exiting the outer wall of the bending section are greatly reduced. Since the pull wire locating ring 1 is made of a flexible material, the pull wire locating ring is difficult to be damaged by frequent pulling during the usage process. The usage stability and reliability are significantly improved. On the other hand, even if one or more pull wire locating rings 1 fall off or are damaged, the pulling easiness of the pull wire is not affected. The condition that the pull wire of the metal bending section is stuck in some pull wire locating ring is avoided.

In a preferred embodiment, the two pull wire perforations 3 on the pull wire locating ring 1 have an axis line parallel to the axis line of the pull wire locating ring 1.

In a preferred embodiment, the axis line of the pull wire locating ring 1 is intersected with a connection line used for connecting the axis lines of the two pull wire perforations 3.

In a preferred embodiment, each of the pull wire locating rings 1 has one end surface provided with two clamping lugs 6 convexly, and has the other end surface provided with two clamping lug seats 7 convexly; the two clamping lugs 6 are provided with a round rod 8 respectively, and the two clamping lug seats 7 define a through hole 9 respectively; the round rods 8 on the two clamping lugs 6 of one pull wire locating ring 1 are inserted into the through holes 9 on the two clamping lug seats 7 of one adjacent pull wire locating ring 1 respectively; and the round rods 8 and the through holes 9 are in one-to-one correspondence, thereby realizing the hinging between the two adjacent pull wire locating rings 1.

In a preferred embodiment, the end surface of the pull wire locating ring 1 provided with clamping lugs 6 defines a notch 5, and the end surface of the pull wire locating ring 1 provided with clamping lug seats 7 is a plane.

In a preferred embodiment, the end surface of the pull wire locating ring 1 provided with clamping lugs 6 is a plane, and the end surface of the pull wire locating ring 1 provided with clamping lug seats 7 defines a notch 5.

In a preferred embodiment, the notch 5 is shaped as a concaved curved surface.

In a preferred embodiment, the pull wire locating ring 1 is made of a medical plastic.

The above embodiments merely illustrate several implementations of the present disclosure. Although the description is specific and detailed, the protection scope of the present disclosure is not limited to the above embodiments. It should be noted that, for the ordinary skill in the art, various variations and improvements also can be made without departing from the idea of the present disclosure. These variations and improvements are intended to be included in the scope of protection of the present disclosure. Therefore, the scope of protection of the present disclosure should be based on the claims appended herein.

What is claimed is:

1. A bending section for a disposable endoscope, comprising a plurality of pull wire locating rings, which are made of a flexible material, wherein
each of the pull wire locating rings has two opposing side walls defining a pull wire perforation therein respectively, the pull wire perforation running through the side wall;
the pull wire locating rings are hinged end to end in series to form a bendable flexible tube structure; the pull wire perforations in the pull wire locating rings are communicated in sequence to define a pull wire channel located in the side walls of the pull wire locating rings; and
the pull wire locating ring has one end surface defining a notch, the notch being a gap reserved for two adjacent pull wire locating rings to be capable of rotating relative to each other.

2. The bending section for a disposable endoscope according to claim 1, wherein the two pull wire perforations on the pull wire locating ring have an axis line parallel to the axis line of the pull wire locating ring.

3. The bending section for a disposable endoscope according to claim 1, wherein the axis line of the pull wire locating ring is intersected with a connection line used for connecting the axis lines of the two pull wire perforations.

4. The bending section for a disposable endoscope according to claim 1, wherein each of the pull wire locating rings has one end surface provided with two clamping lugs convexly, and has the other end surface provided with two clamping lug seats convexly; the two clamping lugs are provided with a round rod respectively, and the two clamping lug seats define a through hole respectively; the round rods on the two clamping lugs of one pull wire locating ring are inserted into the through holes on the two clamping lug seats of one adjacent pull wire locating ring respectively; and the round rods and the through holes are in one-to-one correspondence, thereby realizing the hinging between the two adjacent pull wire locating rings.

5. The bending section for a disposable endoscope according to claim 1, wherein the end surface of the pull wire locating ring provided with clamping lugs defines a notch, and the end surface of the pull wire locating ring provided with clamping lug seats is a plane.

6. The bending section for a disposable endoscope according to claim 1, wherein the end surface of the pull wire locating ring provided with clamping lugs is a plane, and the end surface of the pull wire locating ring provided with clamping lug seats defines a notch.

7. The bending section for a disposable endoscope according to claim 1, wherein the notch is shaped as a concaved curved surface.

8. The bending section for a disposable endoscope according to claim 1, wherein the pull wire locating ring is made of a medical plastic.
